(19) United States
(12) Patent Application Publication
(10) Pub. No.: US 2009/0281409 A1
(43) Pub. Date: Nov. 12, 2009

(54) REINFORCED MEDICAL DEVICE

(76) Inventor: Jeryle WALTER, Valencia, CA (US)

Correspondence Address: COOLEY GODWARD KRONISH LLP;ATTN: PATENT GROUP, Suite 1100, 777 - 6th Street, NW, WASHINGTON, DC, 20001, US

(21) Appl. No.: 12/115,621

(22) Filed: May 6, 2008

(51) Int. Cl. A61B 5/04; A61N 1/00

(52) U.S. Cl. 600/373; 600/372; 607/118

(57) ABSTRACT

An apparatus and method for a medical implant having a reinforced medical implant body. In an embodiment of the invention, an apparatus includes a sheath, a conductive element, a distal electrode and a length limiting element. The conductive element has a proximal end and a distal end. The distal electrode can be coupled to the distal end of the conductive element. The sheath has a distal end, a proximal end and a length defined between the distal end and the proximal end. The sheath of the apparatus is configured to enclose at least a portion of the conductive element. The length limiting element has a distal end and a proximal end. The distal end of the length limiting element can be coupled to the distal end of the sheath and the proximal end of the length limiting element can be coupled to the proximal end of the sheath.

### BACKGROUND

This invention relates generally to medical implants, and more particularly, to a medical implant having a reinforced body.

Implantable medical devices can include a distal electrode that is located near the target site of a nerve and/or muscle. In some instances, after the medical implant has been implanted within the body of a patient, the medical implant requires repositioning or complete removal. The axial forces involved in either one of the two scenarios can have a negative impact on the medical implant. This is especially true in instances when the medical implant is fully encapsulated within the body tissue. For example, an axial force greater than the construction limits the medical implant is designed to withstand may be needed to free the medical implant from the body tissue. In these instances, it is possible that a portion of the medical implant could break, leaving a remaining portion of the medical implant still embedded within the body tissue. Removing the remaining portion of the medical implant could require cutting back the affected area, leading to a greater risk of infection and patient discomfort.

What is needed is a medical implant with a reinforced medical implant body that can withstand greater axial forces to prevent medical implants from breaking during the removal or repositioning process.

### SUMMARY

In an embodiment of the invention, an apparatus includes a conducting element, a distal electrode, a sheath and a length limiting element. The sheath has a proximal end, a distal end and a length which is defined between the distal end and the proximal end. The sheath can be configured to enclose at least a portion of the conductive element, which includes a proximal end and a distal end. The distal electrode can be coupled to the distal end of the conductive element. The length limiting element has a proximal end and a distal end. The proximal end of the length limiting element can be coupled to the proximal end of the sheath and the distal end of the length limiting element can be coupled to the distal end of the sheath. Additionally, the length limiting element can be configured to limit the length of the sheath under tension.

A method according to an embodiment of the invention includes coupling a distal electrode to a distal end of a conductive element, coupling a distal end of a length limiting element to a distal end of a sheath and coupling a proximal end of the length limiting element to a proximal end of the sheath. The sheath can be configured to enclose at least a portion of the conductive element and at least a portion of the length limiting element.

### BRIEF DESCRIPTION OF THE DRAWINGS

FIG. 1 is a schematic illustration shown as an exploded view of an apparatus according to an embodiment of the invention.

FIG. 2 is a cross-sectional illustration of an apparatus according to an embodiment of the invention.

FIG. 3 is a cross-sectional illustration of an apparatus according to an embodiment of the invention with a portion of a length limiting element extending beyond a sheath.

FIG. 4 is a cross-sectional illustration of an apparatus according to an embodiment of the invention with a distal contact portion of a length limiting element being frayed.

FIG. 5 is a cross-sectional illustration of an apparatus according to an embodiment of the invention with a distal contact portion of a length limiting element being knotted.

FIG. 6 is a cross-sectional illustration of an apparatus according to an embodiment of the invention with a portion of a length limiting element embedded within a sheath.

FIG. 7 is a cross-sectional illustration of an apparatus according to an alternative embodiment of the invention with a portion of a length limiting element embedded within a sheath.

FIG. 8 is a cross-sectional illustration of an apparatus according to an embodiment of the invention with a distal retention member and a proximal retention member.

FIG. 9 is a cross-sectional illustration of an apparatus according to an embodiment of the invention with a length limiting element having a length greater than a length of a sheath.

FIG. 10 is a flow chart of a method according to an embodiment of the invention.

### DETAILED DESCRIPTION

In some embodiments, an apparatus includes an medical implant that is configured to be implanted within a body of a patient. The medical implant includes a conductive element, a distal electrode, a sheath and a length limiting element. The conductive element has a distal end and a proximal end. The distal electrode is coupled to the distal end of the conductive element and is configured to stimulate a targeted site within the body. The sheath of the apparatus has a proximal end and a distal end and is configured to enclose at least a portion of the conductive element. The length limiting element has a proximal end and a distal end. The proximal end of the length limiting element is coupled to the proximal end of the sheath and the distal end of the length limited element is coupled to the distal end of the sheath. The length limiting element is configured to limit the length of the sheath under tension.

In some embodiments, a method includes coupling a distal electrode to a distal end of a conductive element, coupling a distal end of a length limiting element to a distal end of a sheath and coupling a proximal end of the length limiting element to a proximal end of the sheath. The sheath can be configured to enclose at least a portion of the conductive element and at least a portion of the length limiting element. In some embodiments, the method includes coupling a proximal electrode to a proximal end of the conductive element. In other embodiments, the method includes coupling a first retention member to the distal end of the sheath and coupling a second retention member to the proximal end of the sheath. The distal end of the length limiting element is coupled to the first retention member and the proximal end of the length limiting element coupled to the second retention member.

As used in this specification, the words “proximal” and “distal” refer to the direction closer to and away from, respectively, an operator (e.g., surgeon, physician, nurse, technician, etc.) who would use a medical implant during a procedure. For example, the end of a medical implant first to contact and/or be inserted into the patient's body would be the distal end, while the opposite end of the medical implant (e.g., the end of the medical implant being operated by the operator or the end of the medical implant last to be inserted into the patient's body) would be the proximal end of the medical implant. Therefore, the distal end of the medical implant is referred to as distal, and the proximal end of the medical implant is referred to as proximal.

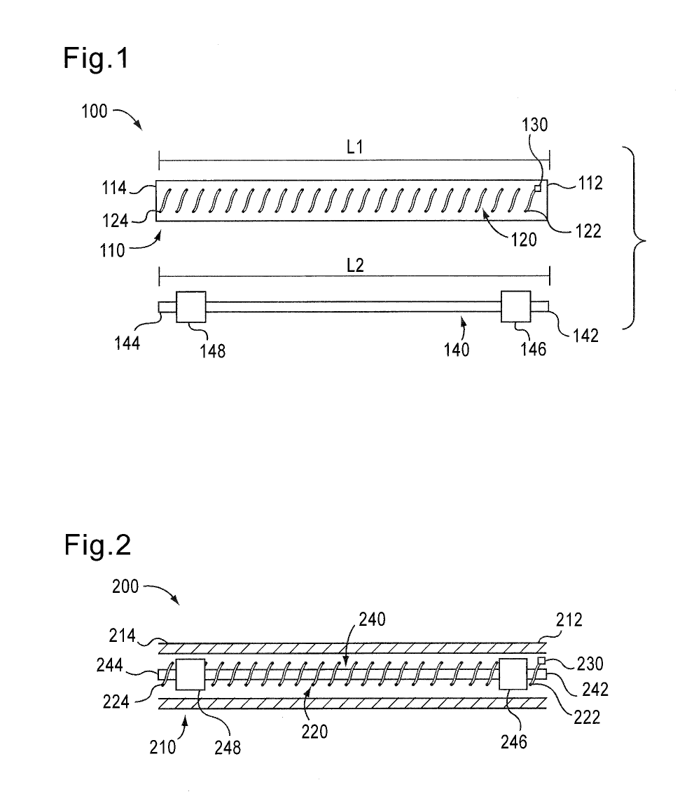

A medical implant has a length limiting element configured to limit the overall length of the medical implant under tension. FIG. 1 is a schematic illustration of a medical implant 100 that includes a sheath 110, a conductive element 120, a distal electrode 130 and a length limiting element 140. The conductive element 120 includes a proximal end 124 and a distal end 122. The distal electrode 130 is coupled to the distal end 122 of the conductive element 120. The sheath 110 includes a proximal end 114, a distal end 112 and a length, L1, defined by the distance between the distal end 112 and the proximal end 114 of the sheath 110. Additionally, the sheath 110 is configured to enclose at least a portion of the conductive element 120. The length limiting element 140 includes a proximal end 144, a distal end 142 and a length, L2, defined by the distance between the distal end 142 and the proximal end 144 of the length limiting element 140. The proximal end 144 of the length limiting element 140 includes a proximal contact portion 148 and the distal end 142 of the length limiting element 140 includes a distal contact portion 146. The proximal contact portion 148 is coupled adjacent to the proximal end 114 of the sheath 110 and the distal contact portion 146 is coupled adjacent to the distal end 112 of the sheath 110.

The length limiting element 140 is affixed to the sheath 110 when the contact portions 146 and 148 of the length limiting element 140 are coupled to the respective ends 112 and 114 of the sheath 110. In this configuration, the length limiting element 140 provides additional reinforcement to the sheath 110 by limiting the length, L1, that the sheath 110 can stretch under tension. For example, when the ends 112 and 114 of the sheath 110 are under tension, the overall length, L1, that the sheath 110 can stretch is approximately the maximum length, L2, of the length limiting element 140.

The restriction that the length limiting element 140 places on the sheath 110 can be beneficial in instances when the removal or repositioning of the medical implant 100 is required. For example, during the removal process the surgeon must place an axial force on the medical implant 100 to detach it from the body tissue. Without the length limiting element 140, the length, L1, of the sheath 110 will continue to increase until it has reached the maximum length determined by the material of the sheath 110. As previously discussed, allowing the sheath 110 to lengthen to capacity can lead to the sheath 110 breaking. However, when the length limiting element 140 is fixed to the sheath 110, the length, L1, that the sheath 110 can extend is limited by the length, L2, of the length limiting element 140. By limiting the length, L1, of the sheath 110 with the length limiting element 140, the overall durability of the medical implant 100 is increased.

In FIG. 1, the length, L1, of the sheath 110 and the length, L2, of the length limiting element 140 are substantially the same. Although the length limiting element 140 is shown as a fixed length, the material of the length limiting element 140 may be slightly elastic. As a result, when the length limiting element 140 is fixed to the sheath 110, both the length, L1, of the sheath 110 and the length, L2, of the length limiting element 140 will increase together under tension. Similarly, the lengthening of both the sheath 110 and the length limiting element 140 will halt together when the length limiting element 140 reaches its maximum length.

FIG. 2 is a cross-sectional illustration of a medical implant 200 that includes a sheath 210, conductive element 220, a distal electrode 230 and a length limiting element 240. The conductive element 220 includes a proximal end 224 and a distal end 222. The distal electrode 230 is coupled to the distal end 222 of the conductive element 220. The sheath 210 also includes a proximal end 214 and a distal end 212 and is configured to at least partially enclose the conductive element 220. The length limiting element 240 includes a proximal end 244, a distal end 242, a proximal contact portion 248 and a distal contact portion 246. The proximal contact portion 248 is coupled adjacent to the proximal end 214 of the sheath 210 and the distal contact portion 246 is coupled adjacent to the distal end 212 of the sheath 210.

The length limiting element 240 is completely enclosed within the sheath 210, as shown in FIG. 2. In some embodiments, however, the sheath 210 can be configured to at least partially enclose the length limiting element 240. FIG. 2 also shows the conductive element 220 coiled around the length limiting element 240. Said another way, the conductive element 220 and the length limiting element 240 are substantially coaxial.

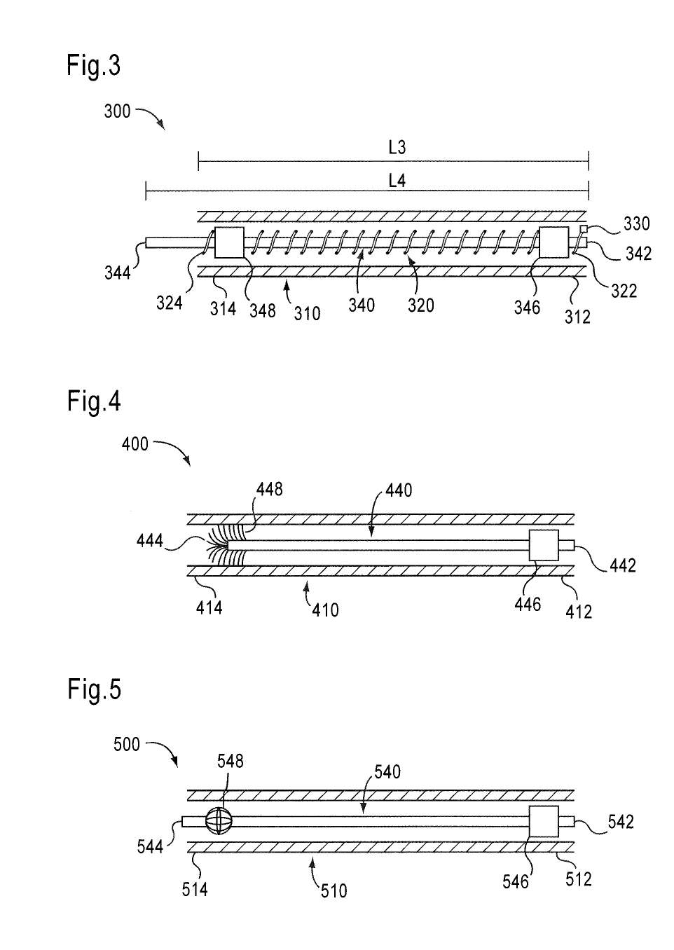

In some embodiments, a length limiting element can have a length greater than a length of a sheath. For example, FIG. 3 is a cross-sectional illustration of a medical implant 300 having a length limiting element 340 with a length, L4, greater than a length, L3, of a sheath 310. The medical implant 300 includes the sheath 310, a conductive element 320, a distal electrode 330 and the length limiting element 340. The conductive element 320 includes a proximal end 324 and a distal end 322. The distal electrode 330 is coupled to the distal end 322 of the conductive element 320. The sheath 310 includes a proximal end 314, a distal end 312 and the length, L3, defined by the distance between the proximal end 314 and the distal end 312. In addition, the sheath 310 is configured to at least partially enclose the conductive element 320. The length limiting element 340 includes a proximal end 344, a distal end 342, a proximal contact portion 348, a distal contact portion 346 and the length, L4, defined by the distance between the proximal end 344 and the distal end 342. The distal contact portion 346 is coupled adjacent to the distal end 312 of the sheath 310 and the proximal contact portion 348 is coupled adjacent to the proximal end 314 of the sheath 310.

The length, L4, of the length limiting element 340 is greater than the length, L3, of the sheath 310. As a result, the proximal end 344 of the length limiting element 340 extends beyond the proximal end 314 of the sheath 310. However, the additional length of the length limiting element 340 does not substantively change the coupling of the length limiting element 340 to the sheath 310. Rather, the additional length of the length limiting element 340 can be beneficial in instances when the removal or repositioning of the medical implant 300 is required. For example, a surgeon can use the additional length at the proximal end 344 of the length limiting element 340 to impart an axial force on the medical implant 300 to help detach the medical implant 300 from the body tissue. As discussed above, the contact portions 346 and 348 of the length limiting element 340 are coupled to the respective ends 312 and 314 of the sheath 310 for the purpose of fixing the length limiting element 340 to the sheath 310. As a result, the length, L3, of the sheath 310 is restricted by the length, L4, of the length limiting element 340 when the axial force is applied. As such, the fixed length limiting element 340 decreases the likelihood that a portion of the sheath 310 will break off and the additional length at the proximal end 344 of the length limiting element 340 makes it easier for the surgeon to hold on to the medical implant when exerting the axial force needed to detach the medical implant 300 from the body tissue.

In some embodiments, the extended portion at the proximal end 344 of the length limiting element 340 can be used to attach the length limiting element 340 to another device. For example, the extended portion at the proximal end 344 of the length limiting element 340 can be attached to a pulling device that would work to help detach the medical implant 300 from the body tissue.

While the contact portions 146, 246, 346, 148, 248 and 348 of the medical implants 100, 200 and 300 have been illustrated as an arbitrary shape in the preceding figures, the contact portions can take any shape or configuration that provides an increased surface area conducive to being coupled to a sheath. For example, FIG. 4 is a cross-sectional illustration of a medical implant 400 with a length limiting element 440 formed from, for example, a braided cable having a frayed end as a proximal contact portion 448. The medical implant 400 includes a sheath 410, a conductive element (not shown), a distal electrode (not shown) and the length limiting element 440. The sheath 410 includes a proximal end 414 and a distal end 412. The length limiting element 440 includes a proximal end 444, a distal end 442, a proximal contact portion 448 and a distal contact portion 446. The proximal contact portion 448 is coupled adjacent to the proximal end 414 of the sheath 410 and the distal contact portion 446 is coupled adjacent to the distal end 412 of the sheath 410.

In the frayed configuration, the proximal contact portion 448 and the proximal end 444 of the length limiting element 440 have an increased surface area. As a result, the area of the proximal contact portion 448 that is coupled to the proximal end 414 of the sheath 410 is increased. Said another way, the increased surface area for coupling the proximal contact portion 448 to the sheath 410 provides a more secure bond between the length limiting element 440 and the sheath 410.

In some embodiments, the contact portions 446 and 448 of the length limiting element 440 are coupled to the sheath 410 using an adhesive. For example, in some embodiments, the length limiting element 440 is coupled to the sheath 410 using a silicone adhesive, such as MED-2000™ or MED-1137™.

Alternatively, a proximal contact portion in a knotted configuration can provide an increased surface area for coupling the proximal contact portion to a proximal end of a sheath. For example, FIG. 5 is a cross-sectional illustration of a medical implant 500 with a length limiting element 540 formed from a 3-0 suture having a proximal contact portion 548 in a knotted configuration. The medical implant 500 includes a sheath 510, a conductive element (not shown), a distal electrode (not shown) and the length limiting element 540. The sheath 510 includes a proximal end 514 and a distal end 512. The length limiting element 540 includes a proximal end 544, a distal end 542, the proximal contact portion 548 and a distal contact portion 546. The distal contact portion 546 is coupled adjacent to the distal end 512 of the sheath 510 and the proximal contact portion 548 is coupled adjacent to the proximal end 514 of the sheath 510.

While only the proximal contact portion 548 of the length limiting element 540 is shown having a knotted configuration, in some embodiments, the distal contact portion 546 can also have a knotted configuration. As a result of both contact portion 546 and 548 being knotted, the durability of the medical implant 500 doubles. Experiments show that a medical implant without a length limiting element has a breaking point of 1.47 pounds, whereas the medical implant 500 with the length limiting element 540 having contact portions 546 and 548 in a knotted configuration has a breaking point of 3.16 pounds when pulled at a rate of 0.6 inches per minute.

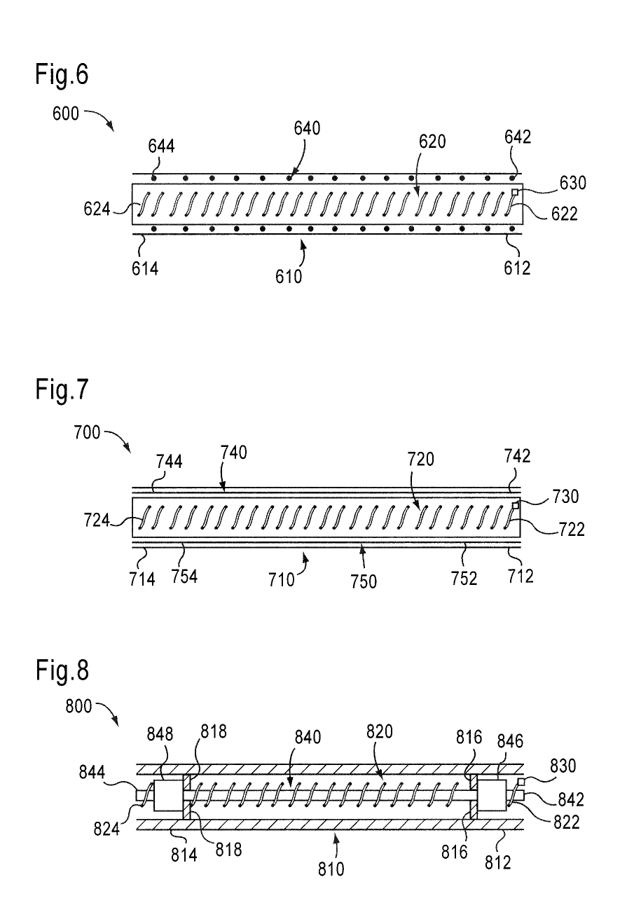

Although embodiments described above have a length limiting element enclosed within a sheath, in some embodiments, a length limiting element is embedded within the wall of a sheath. For example, FIG. 6 is a cross-section illustration of a medical implant 600 having a length limiting element 640 embedded within a sheath 610. The medical implant 600 includes the sheath 610, a conductive element 620, a distal electrode 630 and the length limiting element 640. The conductive element 620 includes a proximal end 624 and a distal end 622. The distal electrode 630 is coupled to the distal end 622 of the conductive element 620. The sheath includes a proximal end 614 and a distal end 612 and is configured to at least partially enclose the conductive element 620. The length limiting element 640 includes a proximal end 644 and a distal end 642. As a result of the length limiting element 640 being embedded within the sheath 610, there is no longer a need for proximal or distal contact portions.

In FIG. 6 the length limiting element 640 is embedded within the sheath 610 in a coiled configuration. In an alternative configuration, however, a length limiting element can be embedded within the wall of a sheath substantially parallel to the longitudinal axis of the sheath 610. Additionally, in some embodiments, a medical implant can include more that one length limiting element. For example, FIG. 7 is a cross-sectional illustration of a medical implant 700 having more than one length limiting element 740 and 750 embedded within a sheath 710 in a linear configuration. The medical implant 700 includes the sheath 710, a conductive element 720, a distal electrode 730, a first length limiting element 740 and a second length limiting element 750. The conductive element 720 includes a proximal end 724 and a distal end 726. The distal electrode 730 is coupled to the distal end 726 of the conductive element 720. The sheath 710 includes a proximal end 714 and a distal end 712 and is configured to enclose at least a portion of the conductive element 720. The first length limiting element 740 and the second length limiting element 750 includes a proximal end 744 and 754 and a distal end 742 and 752, respectively.

Although FIGS. 6 and 7 show the length limiting element 640 and 740 completely embedded within the wall of the sheath 610 and 710, in some embodiments, the length limiting element 640 and 740 is partially embedded within the wall of the sheath 610 and 710.

While previous embodiments describe the contact portions coupled only to the sheath, in some embodiments, the contact portions are coupled to retention members that are coupled to the sheath. For example, FIG. 8 is a cross-sectional illustration of a medical implant 800 with a sheath 810 having a distal retention member 816 and a proximal retention member 818. The medical implant 800 includes a sheath 810, a conductive element 820, a distal electrode 830 and a length limiting element 840. The conductive element 820 includes a distal end 822 and a proximal end 824. The distal electrode 830 is coupled to the distal end 822 of the conductive element 820. The sheath 810 includes a proximal end 814 and a distal end 812. A proximal retention member 818 is coupled adjacent to the proximal end 814 of the sheath 810 and a distal retention member 816 is coupled adjacent to the distal end 812 of the sheath 810. Additionally, the sheath 810 is configured to at least partially enclose the conductive element 820. The length limiting element 840 includes a distal end 842, a proximal end 844, a distal contact portion 846 and a proximal contact portion 848. The distal contact portion 846 is coupled to the distal retention member 816 and the proximal contact portion 848 is coupled to the proximal retention member 818.

The distal retention member 816 and the proximal retention member 818 are configured to fix the length limiting element 840 to the sheath 810. While the retention members 816 and 818 can eliminate the need for coupling the contact portions 846 and 848 of the length limiting element 840 to the sheath 810, in some embodiments, the contact portions 846 and 848 can be coupled to the respective retention members 816 and 818 as well as to the respective ends 812 and 814 of the sheath 810. In instances where the length limiting element 840 is coupled to both the retention members 816 and 818 and the sheath 810, the retention members 816 and 818 provide backup protection. For example, if the length limiting element 840 should decouple from the sheath 810, the retention members 816 and 818 will continue to hold the length limiting element 840 in place.

In some embodiments, the distal retention member 818 and the proximal retention member 816 are made of the same or different material as the sheath 810 and are bonded to the sheath 810. In other embodiments, the sheath 810 and the retention members 818 and 816 are monolithically formed.

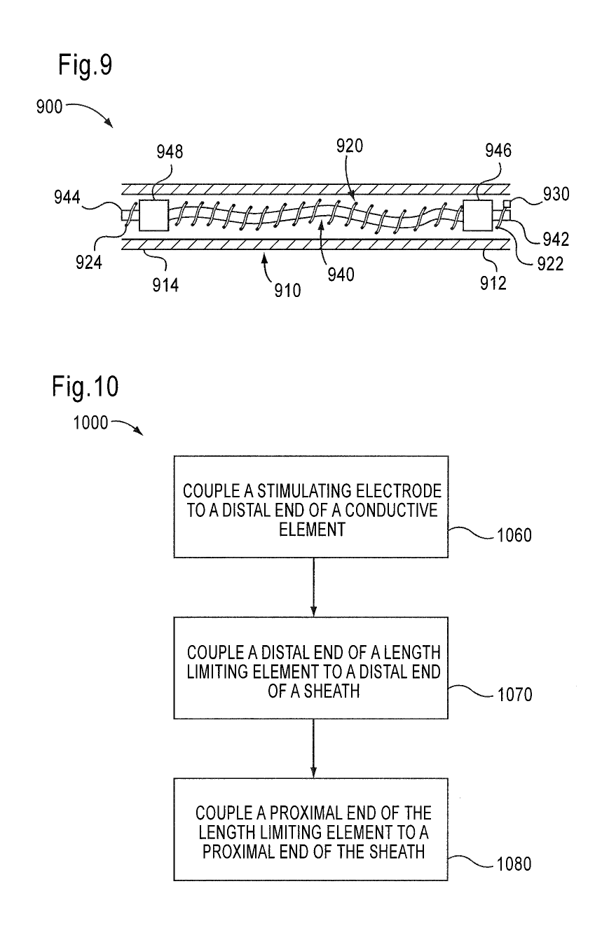

While the embodiments described above show a medical implant in a tensioned state, FIG. 9 is a schematic illustration of a medical implant 900 in a tensionless state having a length limiting element 940 with a length longer than a length of a sheath 910. The medical implant 900 includes the sheath 910, a conductive element 920, a distal electrode 930 and the length limiting element 940. The conductive element 920 includes a distal end 922 and a proximal end 924. The distal electrode 930 is coupled to the distal end 922 of the conductive element 920. The sheath 910 includes a proximal end 914 and a distal end 912. The sheath 910 is configured to at least partially enclose the conductive element 920. The length limiting element 940 includes a distal end 942, a proximal end 944, a distal contact portion 946 and a proximal contact portion 948. The distal contact portion 946 is coupled adjacent to the distal end 912 of the sheath 910 and the proximal contact portion 948 is coupled adjacent to the proximal end 914 of the sheath 910.

In the configuration shown in FIG. 9, the additional length of the length limiting element 940 is located between the contact portions 948 and 946 of the length limiting element 940 and enclosed within the sheath 940. The additional length of the length limiting element 940 allows the length of the sheath 910 to stretch further when the sheath 910 is tensioned.

In some embodiments, as shown in FIG. 10, a method 1000 of manufacturing a medical implant is described. The medical implant includes a sheath, a conductive element, a distal electrode and a length limiting element. The method 1000 includes coupling 1060 the distal electrode to the distal end of the conductive element. The method 1000 includes coupling 1070 a distal end of the length limiting element adjacently to a distal end of the sheath. The method 1000 further includes coupling 1080 a proximal end of the length limiting element adjacently to a proximal end of the sheath.

In some embodiments, the method 1000 includes coupling a proximal electrode to a proximal end of the conductive element. In other embodiments, the method 1000 includes coupling a first retention member to the distal end of the sheath, where the distal end of the length limiting element is coupled to the first retention member and, further, coupling a second retention member to the proximal end of the sheath, where the proximal end of the length limiting element is coupled to the second retention member.

Although FIGS. 2-5, 8 and 9 show the length limiting element being completely enclosed within the sheath, in some embodiments, the sheath may be configured to only partially enclose the length limiting element. In other embodiments, however, the length limiting element can be located outside the sheath and coupled to the outer wall of the sheath.

In FIGS. 2, 3, 8 and 9, the conductive elements 220, 320, 820 and 920 are shown coiled around the length limiting elements 240, 340, 840 and 940 while enclosed within the sheaths 210, 310, 810 and 910. In some embodiments, however, the conductive elements 220, 320, 820 and 920 can be configured to only coil around a portion of the length limiting elements 240, 340, 840 and 940. In other embodiments, the conductive elements 220, 320, 820 and 920 and the length limiting elements 240, 340, 840 and 940 can be woven together in a braided configuration. In yet other embodiments, the conductive elements 220, 320, 820 and 920 and the length limiting elements 240, 340, 840 and 940 are positioned parallel and not coaxially to one another without being interwoven.

Although embodiments described above have a length limiting element in a single configuration (e.g., straight,coiled or serpentine), in some embodiments, the length limiting element can have multiple configurations. For example, a portion of the length limiting element can be in a straight configuration while another portion of the length limiting element can have a coiled configuration.

As described above, a medical implant can include multiple length limiting elements. In some embodiments, each of the length limiting elements are at least partially enclosed within the sheath and include a proximal contact portion and a distal contact portion that can be coupled to the proximal and distal end of the sheath, respectively.

Although embodiments described above have a length limiting element with a length substantially similar to the length of a sheath or, as shown in FIG. 3, the length of the length limiting element with a length greater than the length of the sheath, in some embodiments, the length of the sheath can be greater than the length of the length limiting element. In some embodiments, the length of a length limiting element can be substantially the same as the length of a sheath, but the length limiting element is coupled to the sheath in such a way that a portion of the length limiting element extends beyond the proximal end of the sheath.

In some embodiments, the sheath can be made of an insulative material. For example, the sheath can be made of Teflon™ FEP (DuPont). In other embodiments, the sheath made of Teflon™ FEP can include a nylon coating.

In some embodiments, the length limiting element can be made of a metal. For example, the length limiting element can be a steel cable and/or the like. In other embodiments, the length limiting element can be made of a biocompatible material. Additionally, in some embodiments, the length limiting element can be made of an electrically conductive material. In other embodiments, the length limiting element can be made of a non-conductive material.

In some embodiments, the material of the sheath and the material of the length limiting element can be constructed to have an elastic quality. For example, the elasticity of the sheath can be greater than the elasticity of the length limiting element. Since the length limiting element is coupled to the sheath, under tension the sheath can only stretch as far as the length limiting element can stretch.

Although embodiments described above have a length limiting element with a distal contact portion coupled to a distal end of a sheath and a proximal contact portion coupled to a proximal end of the sheath, in some embodiments, the length limiting element can be coupled to the sheath in a third location. For example, the length limiting element can be coupled to the sheath at a central location in addition to being coupled to the proximal end and the distal end of the sheath. In some embodiments, the length limiting element can further be coupled to a conductive element at a location. In other embodiments, however, the length limiting element can be coupled to the sheath in a location other than the proximal end and distal end of the sheath, and as an alternative to coupling the length limiting element to either the proximal end or distal end of the sheath. For example, the length limiting element can be coupled to the sheath at the proximal end and at a central location of the sheath, but not at the distal end of the sheath.

Although embodiments described above have a proximal contact portion having a frayed and a knotted configuration, respectively, in some embodiments, a distal contact portion can have similar configurations. Additionally, in some embodiments, the contact portion configurations are not limited to frayed or knotted configurations. Rather, the contact portion can be in any configuration where the contact portion has a larger size than the body of the length limiting element in at least one dimension. For example, the contact portions could be in a coiled configuration and/or any like configuration that would increase the surface area of the contact portion. Moreover, in some embodiments, the proximal contact portion and the distal contact portion can have different configurations.

In some embodiments, a distal electrode can be a stimulating electrode, a sensing electrode and/or the like. In some embodiments, a medical implant can include a proximal electrode coupled to a proximal end of a conductive element. The proximal electrode can be, for example, a pickup electrode.

In some embodiments, the medical implant can be an electrical lead. In other embodiments, the medical implant can be an electrical stimulation lead.

While various embodiments of the invention have been described above, it should be understood that they have been presented by way of example only, and not limitation. Thus, the breadth and scope of the invention should not be limited by any of the above-described embodiments, but should be defined only in accordance with the claims and their equivalents.

What is claimed is:

1. An apparatus, comprising:
a conductive element having a distal end and a proximal end;
a distal electrode coupled to the distal end of the conductive element;
a sheath having a distal end and a proximal end and configured to enclose at least a portion of the conductive element, the sheath having a length defined between the distal end and the proximal end of the sheath; and
a length limiting element having a distal end and a proximal end, the distal end of the length limiting element being coupled to the distal end of the sheath and the proximal end of the length limiting element being coupled to the proximal end of the sheath, the length limiting element configured to limit the length of the sheath under tension.

2. The apparatus of claim 1, wherein the length limiting element has a length greater than the length of the sheath under no tension.

3. The apparatus of claim 1, wherein the sheath is elastic.

4. The apparatus of claim 1, wherein the length limiting element is a flexible elongate member.

5. The apparatus of claim 1, wherein at least a portion of the length limiting element is at least partially embedded within a wall of the sheath.

6. The apparatus of claim 1, wherein at least a portion of the length limiting element is substantially straight.

7. The apparatus of claim 1, wherein at least a portion of the length limiting element has a coil configuration.

8. The apparatus of claim 1, further comprising:
a proximal electrode coupled to the proximal end of the conductive element.

9. The apparatus of claim 1, wherein the length limiting element is coupled to a portion of the conductive element.

10. The apparatus of claim 1, wherein the length limiting element includes a biocompatible material.

11. The apparatus of claim 1, wherein the length limiting element is coupled to a portion of the conductive element and a portion of the sheath.

12. The apparatus of claim 1, wherein the length limiting element is braided with at least a portion of the conductive element.

13. The apparatus of claim 1, wherein the length limiting element is a first length limiting element, the apparatus further comprising:
a second length limiting element having a distal end and a proximal end, the distal end of the second length limiting element being coupled to the distal end of the sheath and the proximal end of the second length limiting element being coupled to the proximal end of the sheath.

14. The apparatus of claim 1, wherein the length of the sheath is greater than a length of the length limiting element.

15. The apparatus of claim 1, wherein a portion of the length limiting element is coupled to a portion of the sheath.

16. The apparatus of claim 1, wherein the length limiting element includes a body, the body of the length limiting element having a size and the distal end of the length limiting element having a different size, the size of the distal end of the length limiting element being greater than the size of the body of the length limiting element in at least one dimension.

17. The apparatus of claim 1, wherein the sheath includes an inner wall, the inner wall of the sheath and the distal end of the length limiting element coupled via an interference fit.

18. The apparatus of claim 1, wherein the distal end of the length limiting element is permanently coupled to the distal end of the sheath and the proximal end of the length limiting element is permanently coupled to the proximal end of the sheath.

19. The apparatus of claim 1, wherein the length limiting element is constructed from an elastic material having a first elasticity and the sheath is constructed from an elastic material having a second elasticity, the elasticity of the sheath being greater than the elasticity of the length limiting element.

20. The apparatus of claim 1, further comprising:
a first retention member coupled to the distal end of the sheath, the distal end of the length limiting element being coupled to the first retention member; and
a second retention member coupled to the proximal end of the sheath, the proximal end of the length limiting element being coupled to the second retention member.

21. A method, comprising:
coupling a distal electrode to a distal end of a conductive element;
coupling a distal end of a length limiting element to a distal end of a sheath; and
coupling a proximal end of the length limiting element to a proximal end of the sheath, the sheath configured to enclose at least a portion of the conductive element and at least a portion of the length limiting element.

22. The method of claim 21, further comprising:
coupling a proximal electrode to a proximal end of the conductive element.

23. The method of claim 21, wherein at least a portion of the length limiting element is at least partially embedded within a wall of the sheath.

24. The method of claim 21, wherein the length limiting element is coupled to a portion of the conductive element.

25. The method of claim 21, wherein the length limiting element is coupled to a portion of the conductive element and a portion of the sheath.

26. The method of claim 21, wherein the length limiting element is braided with at least a portion of the conductive element.

27. The method of claim 21, wherein a portion of the length limiting element is coupled to a portion of the sheath.

28. The method of claim 21, wherein the coupling the distal end of the length limiting element to the distal end of the sheath is a permanent coupling, and the coupling the proximal end of the length limiting element to the proximal end of the sheath is a permanent coupling.

29. The method of claim 21, further comprising:
coupling a first retention member to the distal end of the sheath, the distal end of the length limiting element being coupled to the first retention member; and
coupling a second retention member to the proximal end of the sheath, the proximal end of the length limiting element being coupled to the second retention member.
